(19) United States
(12) Patent Application Publication
(10) Pub. No.: US 2022/0263109 A1
(43) Pub. Date: Aug. 18, 2022

(54) ION EXCHANGE MEMBRANE

(71) Applicants: CENTRE NATIONAL DE LA RECHERCHE SCIENTIFIQUE, PARIS (FR); UNIVERSITÉ GRENOBLE ALPES, SAINT-MARTIN-D'HERES (FR); COMMISSARIAT À L'ÉNERGIE ATOMIQUE ET AUX ÉNERGIES ALTERNATIVES, PARIS (FR)

(72) Inventors: Alan LE GOFF, CRETS-EN-BELLEDONNE (FR); Yannig NEDELLEC, Grenoble (FR); Patrice RANNOU, EYBENS (FR); Vincent FORGE, VOUREY (FR); Michael HOLZINGER, SAINT-LAURENT DU PONT (FR)

(73) Assignees: CENTRE NATIONAL DE LA RECHERCHE SCIENTIFIQUE, PARIS (FR); UNIVERSITÉ GRENOBLE ALPES, SAINT-MARTIN-D'HERES (FR); COMMISSARIAT À L'ÉNERGIE ATOMIQUE ET AUX ÉNERGIES ALTERNATIVES, PARIS (FR)

(21) Appl. No.: 17/628,956

(22) PCT Filed: Jul. 29, 2020
(86) PCT No.: PCT/EP2020/071440

§ 371 Date: Jan. 21, 2022

(30) Foreign Application Priority Data
Jul. 30, 2019 (FR) FR1908692

(51) Int. Cl. H01M 8/16; H01M 4/86

(52) U.S. Cl. CPC H01M 8/16; H01M 2008/1095; H01M 4/8605

(57) ABSTRACT

Disclosed is a cell comprising an anode, a cathode and a membrane located between the anode and the cathode, wherein the membrane comprises an aqueous medium and a film comprising amyloid fibers. The invention further relates to said film, stacks of said cells, an electrolyte membrane, and the use of these various devices.

### FIELD OF THE INVENTION

The invention relates to the use of organic molecules, such as proteins, in the form of amyloid fibers in an ion exchange membrane, which membrane can be used in electrochemical devices such as fuel cells.

### PRIOR ART

A fuel cell (FC) is a cell in which an electric voltage is generated through the oxidation on the anode (electron emitter) of a reducing fuel (for example dihydrogen: H₂) coupled with the reduction on the cathode (electron collector) of an oxidant, such as oxygen (O₂) from the air. Proton exchange membrane fuel cells, also known under the name of polymer electrolyte membrane fuel cells (or PEMFC), are a type of fuel cells developed for applications in the field of transport (car, bus, aircraft, etc.) as well as laptops and cell phones. Their specific characteristics include operation in the low pressure ranges (typically atmospheric pressure to 10 bars) and temperatures (typically 20-100° C.) and a specific electrolyte membrane.

For the cell to function, the membrane must be able to conduct the hydroxonium ions (H₃O⁺), also denoted H⁺ in a simplified version, but not the electrons. The membrane must also meet a large number of additional criteria to be able to function. First of all, it must not allow the passage of any gas from one side of the battery cell to the other. This phenomenon is known as “gas crossover.” The membrane must resist the reducing environment at the anode and, at the same time, an oxidizing environment at the cathode. It must also be able to operate within the broadest possible operating humidity and temperature ranges of the PEMFC. Finally, a significant source of energy losses is the resistance of the membrane to the flow of protons. This resistance is minimized by making the membrane as thin as possible (around 50-20 μm). Sulfonated polystyrene membranes were initially used for electrolytes, but were replaced in 1966 by the ionomer Nafion™, which is superior in performance and durability. Polymers comprising heterocyclic units based on poly(pyrrole) and comprising proton acceptor and donor groups are described as capable of forming such membranes in WO2009/040362. To date, Nafion™, a perfluorinated polymer manufactured by Dupont, still remains the reference material for the manufacture of proton exchange membranes. However, other industrial groups (Aciplex, Flemion, 3M, SCC) have developed alternatives (see Kusoglu & A. Z. Weber, Chemical Reviews 117, 987-1104 (2017) [1]).

The operation of a hydrogen-dioxygen cell is particularly clean, since it produces only water and heat and consumes only gases. They are therefore perceived as having a very low impact on the environment. However, the cost of ion exchange membranes, and more particularly of protons (H₃O⁺ ion), still remains a major limiting factor to the development of PEMFC-type FCs. Another problem stems from the inert nature of the perfluorinated materials constituting the membranes such as Nafion™ which is not biodegradable. Finally, an additional problem lies in the moderate performance at low humidity (below 50%) and at low temperatures (below 50° C.). There is therefore a need for ion exchange membranes, at low cost and/or which are biodegradable, and which also exhibit one or more of the above-mentioned characteristics specific to such membranes and performance similar to Nafion™. The aim of the invention is therefore to remedy this need.

### DESCRIPTION OF THE INVENTION

Surprisingly, it has been established by the present application that organic materials, and in particular biological materials, comprising fibers of the amyloid type are able to fully or partially meet these very specific needs.

Amyloid fibers are very stable fibrillar nanostructures formed by a mechanism of spontaneous self-assembly of proteins or polypeptides. These fibers share the same type of intermolecular β-sheet structure. An amyloid protein acquires a secondary structure rich in β-strands that combine via H-bonds to form these β-sheets. The formation of these β-sheets, then of fibers, spontaneously depends on external parameters, in particular the pH and the ionic strength of the medium, the concentration of proteins or polypeptides, the presence of other molecules or further temperature and agitation parameters, which can lead to different fibrillation kinetics and organizations. Functionalized amyloid fibers can be used as electronically conductive nanowires (cf. WO2012/120013). Hydrogels comprising α-lactalbumin are considered for possible use in the biomedical field (dressings) or in paints (cf. WO2012/136909).

In the field of batteries, it is also generally known to use enzymatic proteins at the anode or at the cathode to catalyze oxidation and/or reduction reactions. PCT application WO2008058165 describes such batteries. PCT application WO2009040362, for its part, describes fuel cell proton exchange membranes as an alternative to proton exchange membranes that are already known, such as Nafion™ These alternative membranes include graft polymers comprising a main chain having a heterocyclic unit such as polypyrrol having side chains, or “grafts.” These grafts can comprise peptides or polypeptides of 1 to 10 polypeptide units. Such molecules are not obviously amyloid fibers.

The subject of the invention is an ion exchange membrane, in particular protons, comprising an aqueous liquid and a film comprising amyloid fibers.

A film is a structure having lateral dimensions greatly exceeding its thickness. By “greatly exceeding” it is generally understood that the lateral dimensions are at least 100 times greater than the thickness. This thickness can be advantageously chosen in a range varying from 10 nm to 1 mm, preferably 100 nm to 150 μm, so as to prevent gas crossover while not substantially limiting conduction. A thickness ranging from 1 to 75 μm, in particular 15 to 55 μm (for example 20 to 30 μm) makes it possible to obtain particularly satisfactory results. The surface of the membrane can in turn be chosen in a range from 1 mm² to 10 cm², preferably from 1 to 50 mm². A membrane is a type of film having a structure through which transfer can occur under various driving forces.

Another subject of the invention is a film comprising, or consisting of, amyloid fibers.

The membrane according to the invention comprises such a film itself comprising, or consisting of, amyloid fibers, preferably in a network. It will be recalled that amyloid fibers are generally fibers that result from the self-assembly of proteins or polypeptides. This self-assembly has the characteristic of self-propagating, since the addition of a small quantity (seeding process) of a protein in the form of amyloid fibers in a suspension of this same protein accelerates the growth kinetics of amyloid fibers. Amyloid fibers exhibit a characteristic intermolecular β-sheet structure and also have a characteristic X-ray diffraction profile. Amyloid fibers therefore correspond to the stacking of polypeptides/proteins in linear and generally non-branched fibers. These fibers are stabilized by the stacking of β-strands arranged perpendicular to the axis of the fiber and connected by a network of hydrogen bonds. They usually show Congo red staining associated with birefringence under polarized light (Sipe & Cohen, Journal of Structural Biology 130, 88-98 (2000) [2]) and cause a sharp increase in the fluorescence emitted by thioflavin-T at the wavelength of 480 nm (Sabaté et al., Journal of Structural Biology 162, 387-396 (2008) [3]). Amyloid fibers are generally characterized by a high form factor (“aspect ratio”): diameter from a few nanometers to a few tens of nanometers for a length of the order of a micron up to ten microns when the fibers are formed spontaneously (Doussineau et al., Angewandte Chemie International Edition 55, 2340-2344 (2016) [4]).

In the context of the invention, “amyloid fiber” therefore refers to a fiber comprising, or consisting essentially of at least one polypeptide or at least one protein, said fiber comprising a stack of β-strands of said protein or of said polypeptide, said strands arranged perpendicular to the axis of the fiber being connected by a network of hydrogen bonds. Advantageously, one or more additional structural characteristics mentioned are present, for example their sizes and/or their aspect ratio. The amyloid fibers used in the context of the invention can come from any origin, natural or synthetic. Preferably, they comprise, or consist of, at least one peptide or a protein, and preferably bio-based or of biological origin, for example α-lactalbumin, lysozyme, β-lactoglobulin, prion domain of Het-s and insulin. The use of blends of fibers of different origin is also contemplated, although the use of a single type of fiber has the advantage of simplicity. Advantageously, they are chosen from the group of molecules that are inexpensive and/or available in large quantities, such as α-lactalbumin or lysozyme. It is possible to use a single protein or a mixture of proteins to carry out the invention. Amyloid fibers can also come from polypeptides, or even from peptides.

According to a preferred aspect of the invention, the film and/or the membrane according to the invention is made from a protein solution (which then forms a hydrogel in aqueous medium). After depositing and drying the hydrogel, a film is then obtained, the matrix of which comprises a fibrous network, which comprises, or consists essentially of, amyloid fibers.

Of course, the aqueous liquid allowing the preparation of the hydrogel or that present in the membrane essentially comprises water, but may contain a small proportion of other compounds, such as salts in solution or other additives. The expression “small proportion” may indicate that the liquid consists of at least 80% by mass of water relative to the total mass of liquid, preferably at least 90% by mass of water relative to the total mass of liquid and in particular at least 95% by mass of water relative to the total mass of liquid. Such a hydrogel is generally referred to as a supramolecular gel.

The film and/or the membrane can advantageously be formed by depositing a solution of proteins, the concentration of which is typically from 1 g/L to 500 g/L. Preferably, the concentration of this solution is typically between, or ranging from, 1 g/L and 150 g/L (that is to say, between, or ranging from, 0.1 and 15% by mass proportion relative to the aqueous solvent). The concentration of the protein solution can advantageously range from 25 g/L to 100 g/L.

It is also preferred that the film and/or the membrane according to the invention is self-supporting (or self-supported), that is to say, sufficiently rigid to be able to be handled and placed in a device such as a cell according to the invention. However, according to a variant of the invention, the film and/or the membrane can also comprise a mechanical reinforcement and/or one or more additives. These additives can have one or more objectives and in particular be chosen from the group consisting of:
ions to modulate ionic conduction,
plasticizers to adjust the level of mechanical properties (Young's modulus E [MPa], Lowering of the glass transition) and to facilitate the implementation of the membranes, for example polymers such as methylcellulose, organic and inorganic derivatives with silica base,
crosslinking agents, for example glutaraldehyde (pentane-1,5-dial), to (irreversibly) chemically crosslink the membrane in order to ensure chemical and dimensional stability,
antioxidants and radical traps to limit the chemical degradation processes of the membrane, such as natural antioxidants (e.g. vitamin E (in its 8 natural forms: α-tocopherol, β-tocopherol, γ-tocopherol, δ-tocopherol, α-tocotrienol, β-tocotrienol, γ-tocotrienol and δ-tocotrienol, ascorbic acid, 3,4-dihydroxy-cinnamic acid) or metal cations such as cerium,
UV stabilizers to limit any photo-degradation.

Preferably, the method of manufacturing the film and/or the membrane can comprise a chemical crosslinking step. The crosslinking agent can, for example, be a compound such as glutaraldehyde. The crosslinking step can be carried out by bringing the crosslinking agent together with the film and/or the membrane already formed, for example by exposing said film or said membrane to vapors of the crosslinking agent.

Advantageously, the membrane according to the invention does not allow the passage of electrons. It is also preferred that it does not allow the passage of gas. Advantageously, the membrane should resist the reducing environment (e.g. a medium rich in hydrogen) and, at the same time, an oxidizing environment, such as air (oxygen). Finally, it is also preferred that said membrane can have an ability to exchange ions
at low temperature, for example from 0° C. to 45° C., preferably from 10° C. to 30° C., and in particular around 25° C.; and/or
at low relative humidity, for example from 45% to 75%, preferably from 55% to 65%, and in particular around 60%.

Preferably, it should also be capable of operating at the most common operating temperatures of PEMFCs (45° C. to 95° C.) and 60 to 100% relative humidity, the humidity level being determined in the usual manner.

The membrane allows ion exchange, and in particular the exchange of protons. However, other ions, cations or anions can be exchanged, and in particular hydroxide ions, OH⁻.

Another object of the invention is a cell, preferably a fuel cell, comprising:
an anode;
a cathode; and
a membrane located between the anode and the cathode, said membrane comprising an aqueous liquid and a film comprising amyloid fibers.

Preferably the membrane comprises, or consists of, a membrane such as that described in the present application. The membrane in the cell according to the invention acts as an electrolyte, since it contains the ions that can penetrate and circulate in the matrix of the film by diffusion. Together with the anode and the cathode, the membrane constitutes the heart of the cell.

It is also preferred that the film comprising amyloid fibers is as described in the present application. The basic device comprising an anode, a cathode and a membrane according to the invention can be described as an electrochemical cell, or simply a battery cell.

The anode and the cathode can be of any type, but are generally chosen from the standard type made from materials allowing the electrochemical reactions at the anode and at the cathode. In the case of PEMFCs, they generally consist of a catalyst, for example platinum particles of 2 to 4 nm, of ionic polymer and of a conductive material such as a fabric or a carbon powder. These materials are generally associated with a gas diffusion layer (GDL). This layer makes it possible to ensure a homogeneous distribution of the gases, possibly good management of the water in the cell, and a mechanical strength of the membrane and of the active layers containing the reactive materials of the anode and of the cathode. Such a layer generally consists of a porous carbon fabric with a thickness that may be between 100 μm and 300 μm and coated with polymer, generally PTFE. The carbon fibers of the fabric can be arranged in different ways, for example woven and non-woven.

The cell according to the invention can also comprise additional elements, in particular when the cell according to the invention is a fuel cell (FC), and in particular of the proton-exchange membrane fuel cell (PEMFC) type.

Thus, according to a preferred aspect, the cell according to the invention further comprises two plates:
a first plate for distributing a reducing fuel, for example dihydrogen, and
a second plate for distributing the oxidant and, possibly, for discharging the water.

Each of these plates may be made of, or comprise, machined graphite, metallic materials and/or carbon/polymer or carbon/carbon composites. In addition to their distribution function, the plates can make it possible to ensure the seal between the anode and cathode compartments, possibly to manage the water produced at the cathode, to collect electrons produced at the anode and redistributed at the cathode, to keep the cell within its operating temperature range by virtue of an integrated cooling system and/or to ensure the mechanical cohesion of the stack during clamping and operation.

Another element of the cell according to the invention is the possible presence of sealing means, in particular of seals. These have the function of ensuring the sealing of the battery cell necessary for the optimal and safe operation of the cell and can be made of PTFE, silicone and EPDM (Ethylene propylene diene monomer).

Another object of the invention is also to stack battery cells to form a FC according to the invention as described above. Several battery cells are combined in series to form a stack in order to produce sufficient power for a particular desired application. In this case, the plates are bipolar plates making it possible to carry out this stacking.

Another object of the invention is the use of a material based on amyloid fibers in the manufacture of cells having a single battery cell, cells using a stack of battery cells, and preferably FCs. These cells are in particular the cells described in the present application. Advantageously, the material based on amyloid fibers is a film made up of a fibrous network of proteins, and particularly as described in the present application. A preferred use according to the invention is the manufacture of membranes for cells, and particularly for FCs. In particular, these cells are those according to the invention.

Another object of the invention is a method of manufacturing a film or a membrane according to the invention, characterized in that a gel of amyloid fibers is formed and then spread and dried so as to form said film or said membrane. Preferably, the gel is formed by bringing protein(s) and water into contact under acidic conditions, for example pH 2 to 3, or neutral conditions (for example pH 7 when the protein is insulin), possibly with slight heating (temperature below 80° C.).

Another object of the invention is a device comprising a membrane and/or a cell according to the invention and described in the present application.

Another object of the invention is the use of cells according to the invention for the manufacture of emergency supply devices, portable technologies (computer, mobile phone, charger, etc.) or devices needing a power requirement of less than 100 kW.

Another object of the invention is an electrical device, such as those described above, comprising a cell or a stack of cells according to the invention.

### BRIEF DESCRIPTION OF THE FIGURES

The invention will be better understood upon reading the description which follows, given solely by way of example and with reference to the appended drawings in which:

FIG. 1 is a schematic and partial representation of the PEMFC-type cells of Examples 3 (example according to the invention) and 5 (comparative example).

FIG. 2 shows the polarization and power curves for a PEMFC based on a conventional membrane from Nafion™ and a PEMFC based on a membrane based on α-lactalbumin (α-LAC).

FIG. 3 shows the polarization curves and power curve for a PEMFC based on an α-lactalbumin (α-LAC) membrane and for a PEMFC based on a 95/5 lysozyme/methylcellulose membrane.

### EXAMPLES OF IMPLEMENTATION

### Example 1: Production of a Film Based on α-Lactalbumin According to the Invention

The α-lactalbumin (of bovine origin, CAS number 9051-29-0) was obtained from the company DAVISCO (US) with a purity greater than 90%. These proteins were diluted at a rate of 40 g/L in an aqueous solution of 50 mM hydrochloric acid HCl, to obtain a final pH equal to 2. This suspension was incubated for several days (typically 3 days) at 45° C. with moderate agitation until amyloid fiber formation, which is manifested in the case of α-lactalbumin by the formation of a thixotropic hydrogel. The presence of amyloid fibers was verified by electron microscopy. 0.8 g of the solution was poured dropwise onto a support made of glass fibers coated with PTFE (Techniflon 208 A, 80 μm thick, 53% PTFE by mass, 107 g/m²). Drying was carried out at room temperature in air for 24 hours to form a self-supporting film (20 μm thick).

### Example 2: Production of a Lysozyme-Based Film According to the Invention

Lysozyme (avian origin, CAS number 12650-88-3) from chicken egg white was obtained from Sigma-Aldrich (ref. L-6876) with a purity of approximately 95%. These proteins were diluted at a rate of 40 g/L in an aqueous solution of hydrochloric acid HCl for a final pH of 2.7 containing 90 mM of NaCl. This suspension was incubated for several days (typically 3 days) at 60° C. with moderate agitation until amyloid fiber formation, which is manifested in the case of lysozyme by the formation of a hydrogel. The presence of amyloid fibers was verified by electron microscopy. In this example, 5% by mass of a methylcellulose solution in HCl (pH 3) is added to the lysozyme solution in order to improve the mechanical properties (stability, elasticity) of the film obtained after drying.

0.8 g of the solution was poured dropwise onto a support made of glass fibers coated with PTFE (Techniflon 208 A, 80 μm thick, 53% PTFE by mass, 107 g/m²). Drying was carried out at room temperature in air for 24 hours to form a self-supporting film (20 μm thick).

### Example 3: Production of Fuel Cells

Cells according to the invention were each produced with the membranes of Examples 1 and 2. For each cell, a membrane 30 was detached from its respective support and was positioned between two electrodes 20 of a conventional test fuel cell (hydrogen) from the company Paxitech (France). In summary, a hydrogen/air fuel cell having 5 cm² of active surface.

Commercial gas diffusion electrodes are placed on a Sigracet 29 BC brand gas diffusion layer (purchased from Fuelcellstore (USA)). It is a non-woven carbon paper with a microporous layer (MPL) treated with 5% by weight PTFE. It has a total thickness of 235 μm (microns). The electrodes thus comprise a 0.5 mg·cm⁻² platinum charge on a carbon powder support of the Vulcan type deposited on carbon fiber paper (Sigracet 29BC).

The electrodes themselves are positioned on outer graphite plates 10 machined with a serpentine gas flow. That is, the active surface comprises a serpentine shaped recess 1 mm wide by 1 mm deep (not shown).

PTFE gaskets and sub-gaskets are used to prevent gas leakage and to ensure adequate electrical insulation.

### Example 4: Battery Performance According to the Invention

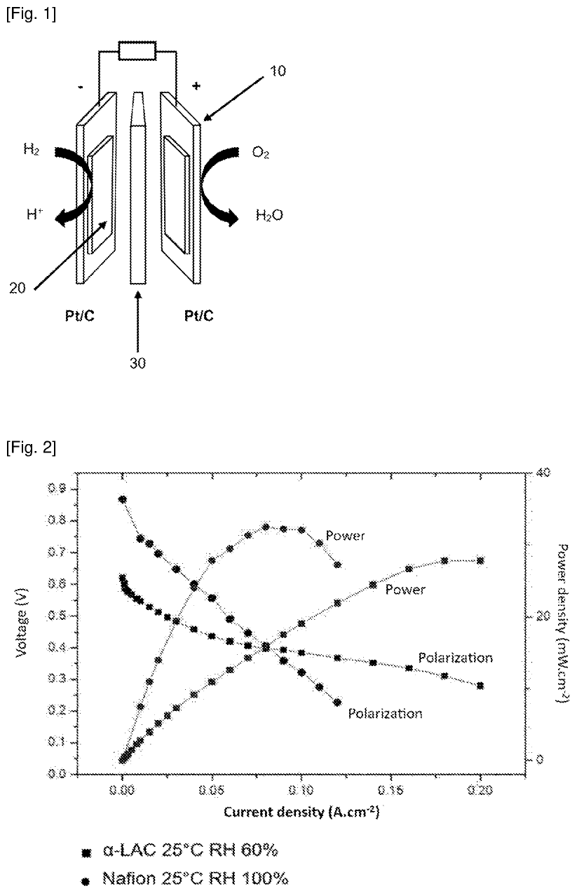

In operation, the hydrogen (H₂) enters through the plate 10 on the left in FIG. 1. Once it has arrived at the anode, the hydrogen dissociates (oxidation) in H⁺ ions and in electrons according to: 2H²=4H⁺+4e⁻. The ions then pass through the membrane 30, but the electrons, blocked, are forced to take the external circuit, which generates current. At the cathode, the hydrogen ions, electrons and oxygen (pure or from air) meet to form water according to the reaction: 4H⁺+4e⁻+O₂=2H₂O. The water and oxygen pass through the right plate 10. This reaction will also produce heat that can be recovered.

FIG. 3 shows the polarization and power curves that were obtained by galvanostatic discharges of 30 s at room temperature under atmospheric pressure with humidified gases (minimum relative humidity of 60% RH) (H₂ and air) with respective flow rates of 20 mL min-1 for a membrane based on lysozyme and α-lactalbumin.

These results show that a membrane comprising a film of amyloid fibers is also a good proton conductor. The lysozyme-based membrane compared to α-lactalbumin leads to slightly lower performance (7 mW cm⁻² at 0.4 V). Polarization curves and power curve for a PEMFC based on an α-lactalbumin (α-LAC) membrane and for a PEMFC based on a 95/5 lysozyme/methylcellulose membrane. The discharges were carried out at 1 atm in H₂ and air at a humidity level of 60%.

### Comparative Example 5: Production of a Cell with Nafion™ Membrane

To demonstrate the advantages of the membranes according to the invention, comparative tests were carried out. The only difference between the devices is the use of a membrane 30 with the following characteristics (DUPONT Nafion™ NRE212, thickness 50 μm—CAS No. 31175-20-9) instead of a membrane (30) according to the invention. The tests were carried out under conditions identical to those described above except that the discharges were carried out at a humidity level of 100% and not of 60%.

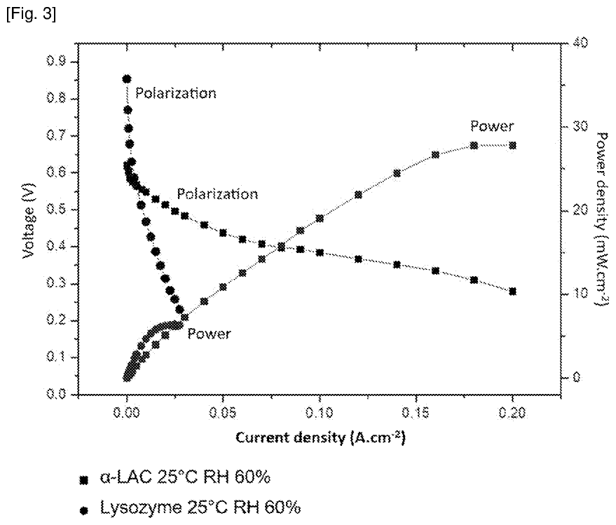

FIG. 2 shows the polarization curve (black) and the power curve (blue) the PEMFC batteries based on a conventional membrane made from Nafion™ and a PEMFC based on an α-lactalbumin (α-LAC). The discharges were carried out at 1 atm in H₂ and air at a humidity level of 60% for α-lactalbumin and 100% for Nafion™.

The performances obtained at 25° C. (22 mW cm-2 at 0.4 V) show that the α-LAC-based membrane is an excellent proton conductor and is able to approach the performance of Nafion™ (32 mW cm-2 at 0.4 V) under these conditions (25° C., RH 60%).

### Example 6: Production of a Crosslinked Film Based on α-Lactalbumin and Glutaraldehyde According to the Invention

The self-supported protein membranes were also subjected to a chemical crosslinking step in the presence of glutaraldehyde vapor (Supplier Sigma-Aldrich, 50% (by mass) in water). The protein film of Example 1, once dried, is subjected to glutaraldehyde vapors for 30 min at 25° C.

This crosslinking step allows the self-supported film to be resistant in solution in water at acidic pH (tested pH=3) and up to 80° C. In PEMFC operation, this step therefore allows the cell to operate over a wide temperature range. Its temperature resistance goes from 35° C., without chemical crosslinking, to at least 60° C. after chemical crosslinking, or even more. In addition, a PEMFC comprising such a membrane does not lose its performance after several days of operation.

The invention is not limited to the embodiments described here, and other embodiments will become clearly apparent to a person skilled in the art. It is in particular possible to consider the use of peptides capable of forming amyloid fibers that organize themselves into hydrogels. It is also possible to use the membranes according to the invention on any type of PEMFC. It can be used not only for hydrogen fuel cells, but also direct methanol fuel cells (DMFC).

1. A fuel cell comprising:
an anode;
a cathode; and
a membrane located between the anode and the cathode, said membrane comprising an aqueous liquid and a film comprising amyloid fibers.

2. The fuel cell according to claim 1, wherein said membrane is a proton exchange membrane.

3. The fuel cell according to claim 1, wherein said film has a thickness chosen from a range varying from 10 nm to 1 mm.

4. The fuel cell according to claim 1, wherein the amyloid fibers comprise, or consist of, at least one protein such as α-lactalbumin or lysozyme.

5. The fuel cell according to claim 1, wherein said film further comprises an additive selected from the group consisting of ions, plasticizers, and crosslinking agents.

6. The fuel cell according to claim 1, wherein said cell further comprises two plates:
a first plate for distributing a reducing fuel, for example dihydrogen, and
a second plate for distributing the oxidant and, possibly, for discharging the water.

7. (canceled)

8. (canceled)

9. A fuel cell comprising a stack of at least two fuel cells as described in claim 1.

10. (canceled)

11. An electrical device comprising a fuel cell described in claim 1.

12. A method of manufacturing a film or membrane based on amyloid fibers, comprising forming a gel of amyloid fibers, and spreading and drying the gel so as to form said film or said membrane.

13. The method according to claim 12, wherein said gel of amyloid fibers is formed by contacting protein(s) and/or polypeptides with water under conditions allowing formation of amyloid fibers.

14. The method according to claim 13, wherein said protein is α-lactalbumin or lysozyme.

15. The fuel cell according to claim 5, wherein the crosslinking agents are selected from glutaraldehyde, antioxidants, radical traps, or UV stabilizers.

16. An electrical device comprising a fuel cell described in claim 9.

17. The method of claim 12, wherein the gel is spread and dried on a solid support.
